(19) United States
(12) Patent Application Publication
(10) Pub. No.: US 2016/0051429 A1
(43) Pub. Date: Feb. 25, 2016

(54) MEDICAL EQUIPMENT TABLE AND MEDICAL EQUIPMENT

(71) Applicant: GE MEDICAL SYSTEM GLOBAL TECHNOLOGY COMPANY, LLC, Waukesha, WI (US)

(72) Inventors: Koji YANAGIHARA, Tokyo (JP); Akira MIZUKOSHI, Tokyo (JP)

(21) Appl. No.: 14/779,281

(22) PCT Filed: Mar. 20, 2014
(86) PCT No.: PCT/US14/31308

§ 371 Date: Sep. 22, 2015

(30) Foreign Application Priority Data
Mar. 22, 2013 (JP) 2013-060429

(51) Int. Cl. A61G 7/053; A61B 6/04

(52) U.S. Cl. CPC A61G 7/053; A61B 6/0442; A61G 2007/051

(57) ABSTRACT

A medical equipment table having a placement surface on which a subject is placed. The medical equipment table is equipped with door structures provided at side faces extending along a longitudinal direction of the medical equipment table. Each of the door structures has a door section, a horizontal axis rotation lock mechanism enabling horizontal axis rotation with one end in a vertical direction of the door section being taken as a support axis, and locks at a plurality of horizontal axis rotating positions, and a vertical axis rotation lock mechanism enabling vertical axis rotation with one end in a horizontal direction of the door section being taken as a support axis, and locks at a plurality of vertical axis rotating positions.

### CROSS-REFERENCE TO RELATED APPLICATIONS

This is a national stage application under 35 U.S.C. §371(c) of prior filed, co-pending PCT application serial number PCT/US2014/031308, filed on Mar. 20, 2014, which claims priority to Japanese Patent Application Serial No. 2013-060429 filed Mar. 22, 2013 and titled MEDICAL EQUIPMENT TABLE AND MEDICAL EQUIPMENT. The above-listed applications are herein incorporated by reference.

### BACKGROUND OF THE INVENTION

Embodiments of the invention relate to a medical equipment table for placing a subject thereon, and medical equipment having the table.

As a medical equipment table, there has heretofore been proposed a table in which an inverted U-shaped handrail is provided at its side face rotatably around a horizontal axis and can be fixed by changing it upward or downward as referred to in Japanese Patent Application No. 2011-92545.

According to such a table, when a subject gets on and off the table, the handrail is directed downward, thereby making it possible to prevent it from obstructing the getting on/off. When the handrail is needed when a subject low in athletic ability lies on the table and stands up, for example, it is possible to easily cope with it by directing the handrail upward.

### SUMMARY OF THE INVENTION

In the above table, however, the handrail must always be directed downward when the subject gets on and off the table. It is therefore not possible for the subject to grasp the handrail even when the subject wants to grasp the handrail when the subject gets on and off the table. For example, when a subject is being placed on a wheelchair or low in athletic ability due to the old age or disorders, there is a need that the subject desires to grasp the handrail when the subject gets on and off the table. Even in such a case, however, the subject is not able to grasp the handrail and needs to take large assistance from an operator or the like. It remains difficult for the subject to get on and off the table.

With such circumstances, there has been a demand for a medical equipment table which allows a subject to get on and off easily regardless of the subject being high or low in athletic ability, and medical equipment having the table.

According to the invention of the above aspects, door structures each having a door section, a horizontal axis rotation lock mechanism and a vertical axis rotation lock mechanism therefor are provided at the side faces of a table. Therefore, the door section is opened by vertical axis rotation and locked with respect to a subject being placed on a wheelchair or a subject low in athletic ability due to the old age or disorders, thereby making it possible to allow the subject to get on and off the table while grasping the door section. On the other hand, the door section is opened by vertical axis rotation and lowered down by horizontal axis rotation with respect to a subject high in athletic ability, thereby making it possible to allow the subject to get on and off the table without being disturbed by the door section. Thus, the table can be deformed according to the athletic ability of the subject so that the subject can easily get on and off the table. As a result, the subject becomes easy to get on and off the table regardless of being high or low in athletic ability.

### BRIEF DESCRIPTION OF THE DRAWINGS

FIG. 1 is a diagram showing a magnetic resonance imaging system according to an embodiment of the invention;

FIG. 2 is a diagram illustrating a configuration of a table;

FIG. 3 is a first diagram depicting a configuration of a door structure;

FIG. 4 is a second diagram showing the configuration of the door structure;

FIG. 5 is a diagram illustrating a rotating range of a door section;

FIG. 6 is a diagram for describing a first function of a pair of door structures;

FIG. 7 is a diagram for describing a second function of the pair of door structures;

FIG. 8 is a diagram for describing a third function of the pair of door structures;

FIG. 9 is a diagram for describing a fourth function of the pair of door structures; and

FIG. 10 is a diagram showing the flow of movements of a subject riding on a wheelchair and an operator when the subject gets on the table.

### DETAILED DESCRIPTION

An embodiment of the present invention will hereinafter be described.

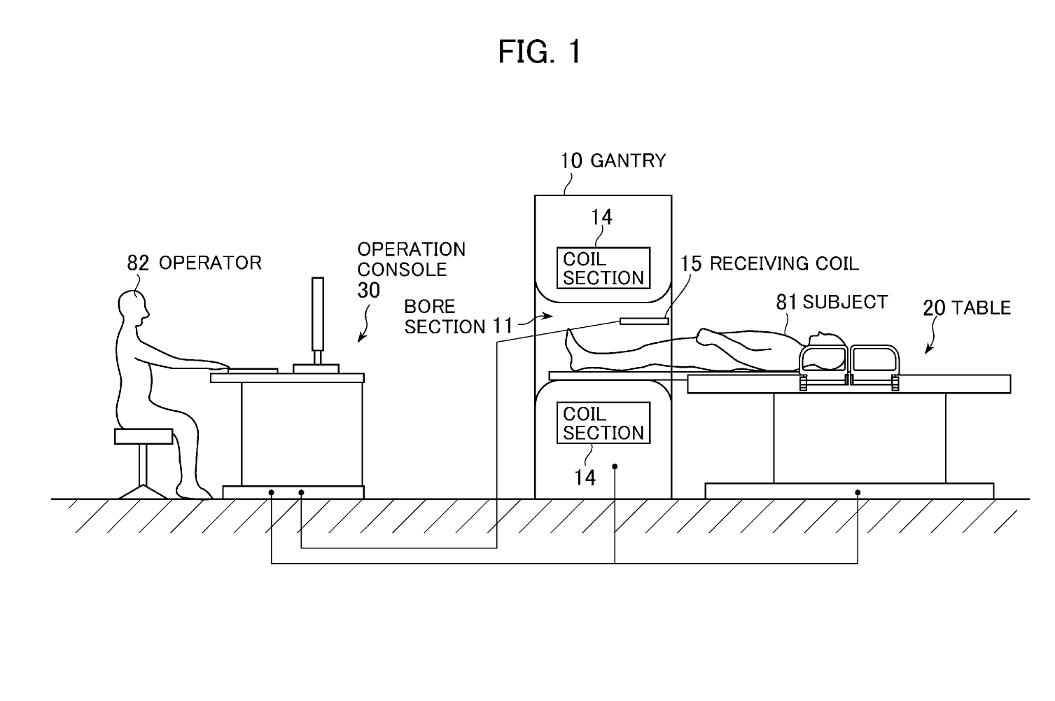

FIG. 1 schematically shows the configuration of a magnetic resonance imaging system (medical equipment) 1 according to the present embodiment. As shown in the same drawing, the magnetic resonance imaging system 1 has a gantry 10, a table 20 and an operation console 30.

The gantry 10 has a bore section 11. A subject 81 is carried in the bore section 11. The gantry 10 forms a static magnetic field within the bore section 11 by a magnet provided thereinside. Further, the gantry 10 drives an internal coil section 14 to generate a gradient magnetic field within the bore section 11. A receiving coil 15 receives magnetic resonance signals generated from the subject 81 and transmits the same to the operation console 30.

The table 20 carries the subject 81 in and out of the bore section 11.

The operation console 30 controls the gantry 10 and the table 20 such that imaging is performed on the subject 81. Further, the operation console 30 receives an output signal from the receiving coil 15 and reconstructs an image, based on the signal.

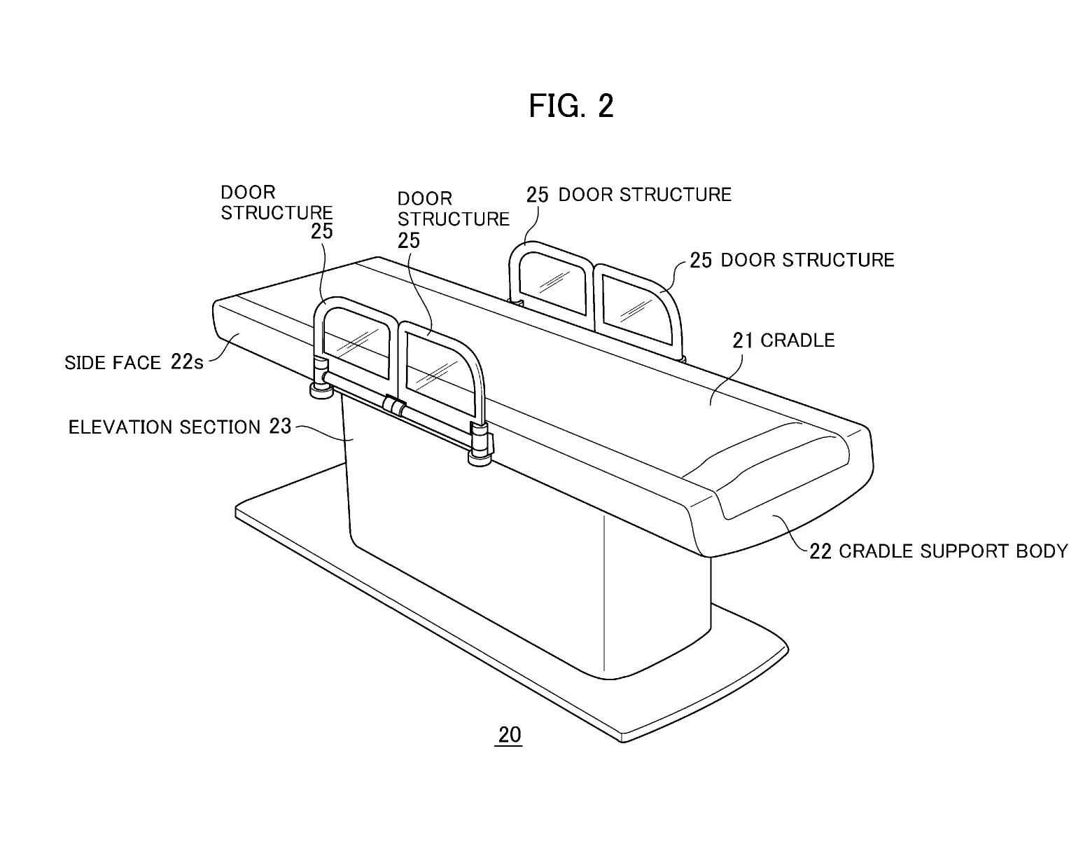

FIG. 2 is a diagram showing the configuration of the table 20. As shown in FIG. 2, the table 20 has a cradle 21 for placing the subject 81 thereon, a cradle support body 22 which supports the cradle 21 so that the cradle 21 is linearly movable horizontally, and an elevation section 23 which elevates the cradle support body 22.

The cradle 21 is formed substantially rectangular as viewed in a vertical direction and has a placement surface for placing the subject 81 thereon. The cradle support body 22 supports the cradle 21 in such a manner that the cradle 21 can be linearly moved horizontally in the direction of the body axis of the subject 81. The elevation section 23 has a driving part (not shown) which vertically elevates the cradle support body 22. The bottom of the elevation section 23 is fixed to the floor. In the present embodiment, the horizontal linear moving direction of the cradle 21 is called a z-axis direction.

Pairs of symmetrical door structures 25 are respectively attached close to each other to both side faces 22s extending along the longitudinal direction of the cradle support body 22 approximately in the middle of the side faces 22 in the z-axis direction.

The configuration of the door structure 25 will hereinafter be described in detail.

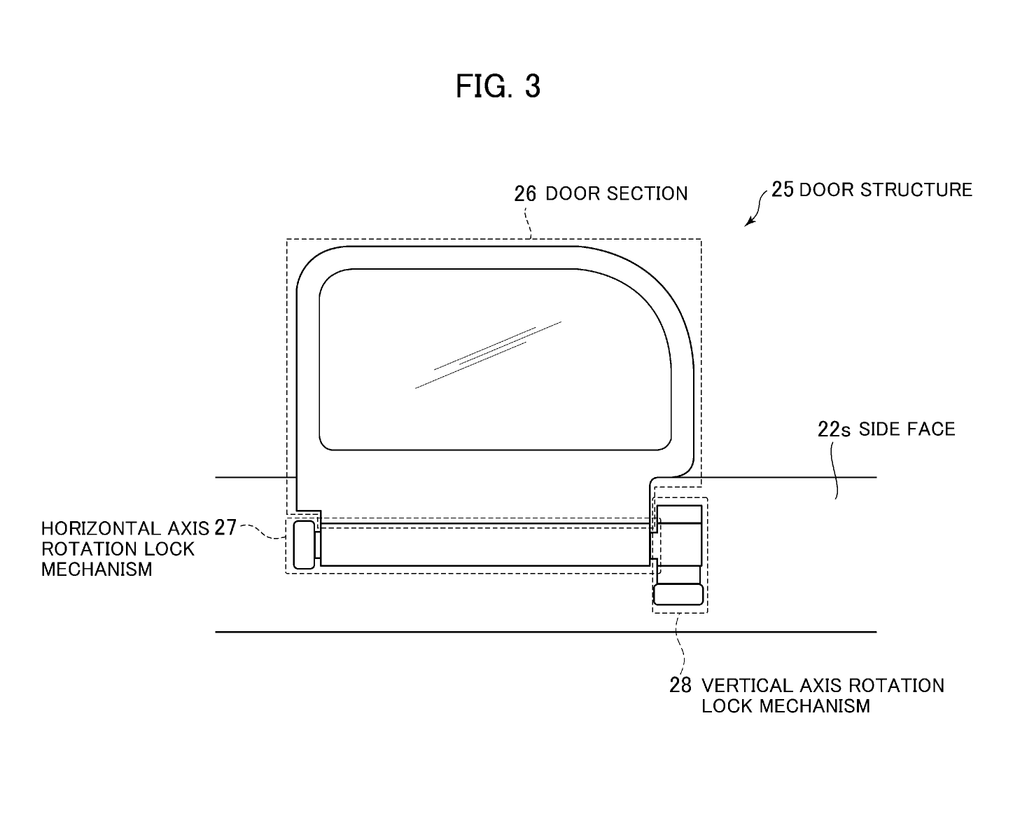

FIG. 3 is a first diagram showing the configuration of the door structure 25. As shown in FIG. 3, the door structure 25 has a door section 26, a horizontal axis rotation lock mechanism 27 which enables horizontal axis rotation with one end in the vertical direction (upper/lower directions in FIG. 3) of the door section 26 being taken as a support axis, and its multistage lock at a plurality of horizontal axis rotating positions, and a vertical axis rotation lock mechanism 28 which enables vertical axis rotation (rotation about a vertical axis) with one end in the horizontal direction (lateral direction in FIG. 3) of the door section 26 being taken as a support axis, and its multistage lock at a plurality of vertical axis rotating positions. The door structure 25 is comprised of a nonmagnetic body. It is comprised of, for example, aluminum, stainless-steel, a plastic resin or the like.

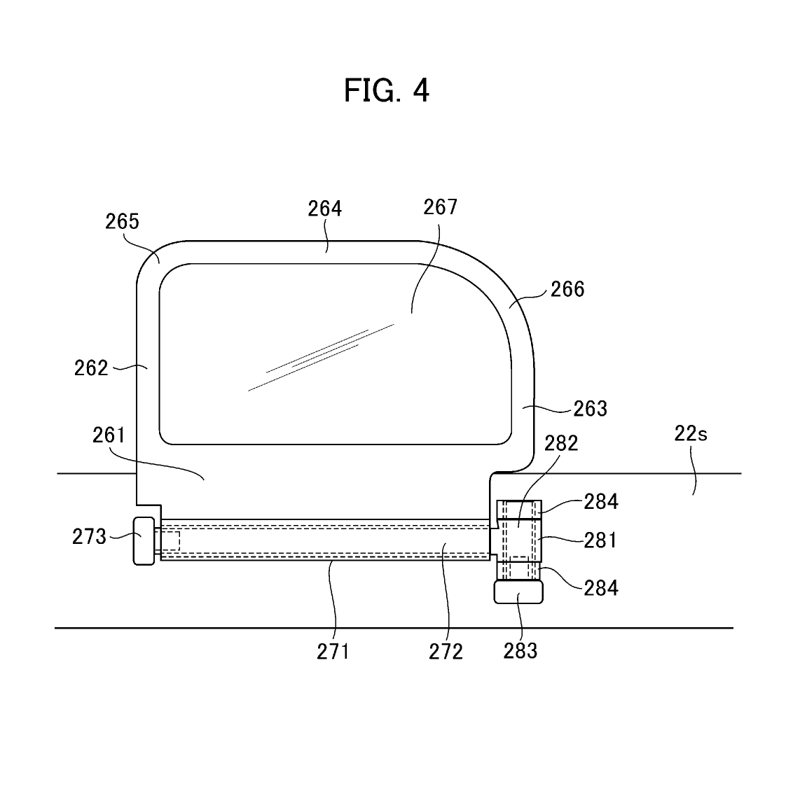

FIG. 4 is a second diagram showing the configuration of the door structure 25. As shown in FIG. 4, the door section 26 has a shape close to a plate shape in which a rectangle of which the horizontal direction is assumed to be a longitudinal direction and the direction perpendicular to the horizontal direction is assumed to be a transverse direction is taken as a plate surface as a whole. The door structure 26 has a plate-like member 261 in which a rectangle of which the two side edges extending parallel to each other along the horizontal direction assume long sides is taken as a plate surface, a first columnar member 262 extending in the short side direction of the plate surface of the plate-like member 261 from one end in the horizontal direction of the plate-like member 261, a second columnar member 263 extending in the short side direction of the plate surface thereof from the other end in the horizontal direction of the plate-like member 261 and in the same direction as the first columnar member 262, a third columnar member 264 extending in parallel to the long side of the plate surface of the plate-like member 261, a first curved member 265 which is curved in an arc shape and couples the first columnar member 262 and the third columnar member 264, and a second curved member 266 which is curved in an arc shape and couples the second columnar member 263 and the third columnar member 264. In an embodiment, the first columnar member 262 through the second curved member 266 are respectively composed of a pipe-like member. Further, the plate-like member 261 through the second curved member 266 are integrally formed.

Further, the door section 26 has a window member 267 arranged to occupy a region surrounded by the plate-like member 261 through the second curved member 266. The window member 267 is comprised of a member having light transmissivity and relatively high in rigidity, e.g., a transparent or non-transparent acrylic plate or the like.

The horizontal axis rotation lock mechanism 27 has a first tubular member 271 connected and fixed along the outer long side portion of the plate-like member 261 and having a cylindrical shape extending in the horizontal direction, a first columnar or cylindrical fitting member 272 which is inserted from an opening on one end side of the first tubular member 271 close to the second columnar member 263 and fitted into the cylinder of the first tubular member 271, and a horizontal axis rotation lock knob 273 formed with a columnar or cylindrical ejection/insertion portion ejected from and inserted in the cylinder of the first tubular member 271 from an opening on the other end side thereof.

The ejection/insertion portion of the horizontal axis rotation lock knob 273 is engaged with the first fitting member 272 inside the cylinder of the first tubular member 271 and capable of being drawn from and inserted into the first tubular member 271 by a guide (not shown). A plurality of concave portions (not shown) are formed on one of the outer periphery of the ejection/insertion portion of the horizontal axis rotation lock knob 273 and the inner periphery on the other end side of the first tubular member 271 at predetermined intervals in the circumferential direction. A plurality of convex portions (not shown) respectively fitted in the concave portions are provided on the other thereof at predetermined intervals. When the ejection/insertion portion of the horizontal axis rotation lock knob 273 is ejected from the first tubular member 271, the convex portions are disengaged from the concave portions so that the first tubular member 271 becomes rotatable about the horizontal axis. On the other hand, when the ejection/insertion portion of the horizontal axis rotation lock knob 273 is inserted in the first tubular member 271, the concave portions are fitted into the concave portions so that the first tubular member 271 is locked at a prescribed horizontal axis rotating position. The horizontal axis rotating position of the door section 26 is determined by a fitting angle between each convex portion and its corresponding concave portion. In an embodiment, the rotation lock mechanism using the convex portions and the concave portions is one example illustrative of a rotation lock mechanism and is not limited to the present embodiment.

The vertical axis rotation lock mechanism 28 has a second tubular member 281 cylindrical in shape extending in the vertical direction, which is connected and fixed to one end in the horizontal direction of the first fitting member 272 and located on the side opposite to the side of the horizontal axis rotation lock knob 273, a second fitting member 282 columnar or cylindrical in shape, which is inserted and fitted inside the tube of the second tubular member 281 from an opening on one end side of the second tubular member 281, a vertical axis rotation lock knob 283 formed with an ejection/insertion portion ejected from and inserted in the tube of the second tubular member 281 from an opening on the other end side thereof, and a support portion 284 which supports the second fitting member 282 and is fixed to the side face 22s of the cradle support body 22.

The vertical axis rotation lock mechanism 28 basically has a mechanism similar to the horizontal axis rotation lock mechanism 27 and enables vertical axis rotation and locking at a predetermined vertical axis rotating position according to the similar mechanism. Thus, in the present embodiment, the description for the vertical axis rotation lock mechanism 28 will be omitted.

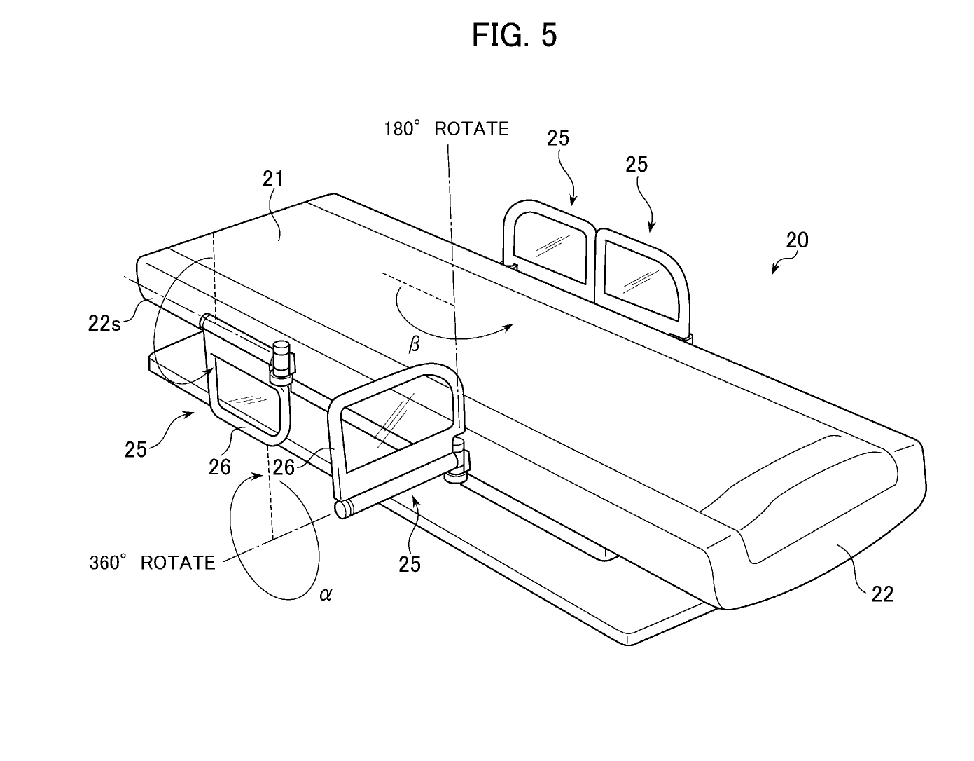

FIG. 5 is a diagram showing the range of rotation of the door section 26.

The door section 26 can be turned at 360° upon the horizontal axis rotation. The door section 26 can be locked at least at a first horizontal axis rotating position where the door section 26 is most upward with respect to the support axis for the horizontal axis rotation and a second horizontal axis rotating position where the door section 26 is most downward with respect to the support axis for the horizontal axis rotation. In the present embodiment, the door section 26 can be locked at 30-degrees intervals and locked at a plurality of horizontal axis rotating positions including a third horizontal axis rotating position where the door section 26 becomes horizontal, in addition to the first horizontal axis rotating position and the second horizontal axis rotating position. Thus, the door section 26 can adopt 0°, 30°, . . . , 300° and 330° assuming that the opening angle (hereinafter called a horizontal axis rotating angle) from the side face 22s of the cradle support body 22 at the horizontal axis rotation is a. In an embodiment, the door section 26 may be locked at 15° intervals or 45° intervals or may be locked at substantially continuous desired horizontal axis rotating positions.

Further, the door section 26 can be rotated by 180° upon the vertical axis rotation and locked at least at a first vertical axis rotating position where the door section 26 is most inward (nearest the center in z-axis direction) with respect to the support axis for the vertical axis rotation, a second vertical axis rotating position where the door section 26 is most outward (closest to the end in the z-axis direction) with respect to the support axis for the vertical axis rotation, and a third vertical axis rotating position where the door section 26 becomes perpendicular to the side face 22s. In the present embodiment, the door section 26 can be locked at 45° intervals and locked at a plurality of vertical axis rotating positions including the first through third vertical axis rotating positions. Thus, the door section 26 can adopt 0°, 45°, 90°, 135° and 180° assuming that the opening angle (hereinafter called a vertical axis rotating angle) from the side face 22s of the cradle support body 22 at the vertical axis rotation is β. In an embodiment, the door section 26 may be locked at 15° intervals or 30° intervals or may be locked at substantially continuous desired vertical axis rotating positions.

The door structure 25 is configured in such a manner that when the door section 26, i.e., the first tubular member 271 is at the first horizontal axis rotating position, the essential part of the door section 26 is located higher than the placement surface of the cradle 21. Further, the door structure 25 is configured in such a manner that when the door section 26 is at the second horizontal axis rotating position, the door section 26 is located lower than the placement surface of the cradle 21.

A plurality of functions that the door structures 25 have will be described below. In the present embodiment, the position where the horizontal axis rotating angle becomes α=0° and the vertical axis rotating angle becomes β=0° is referred to as a home position for each door structure 25.

First Function: Function of Supporting Standing Up from a Wheelchair

The pair of door structures 25 has as the first function, the function of supporting the action of the subject 81 to stand up from the wheelchair.

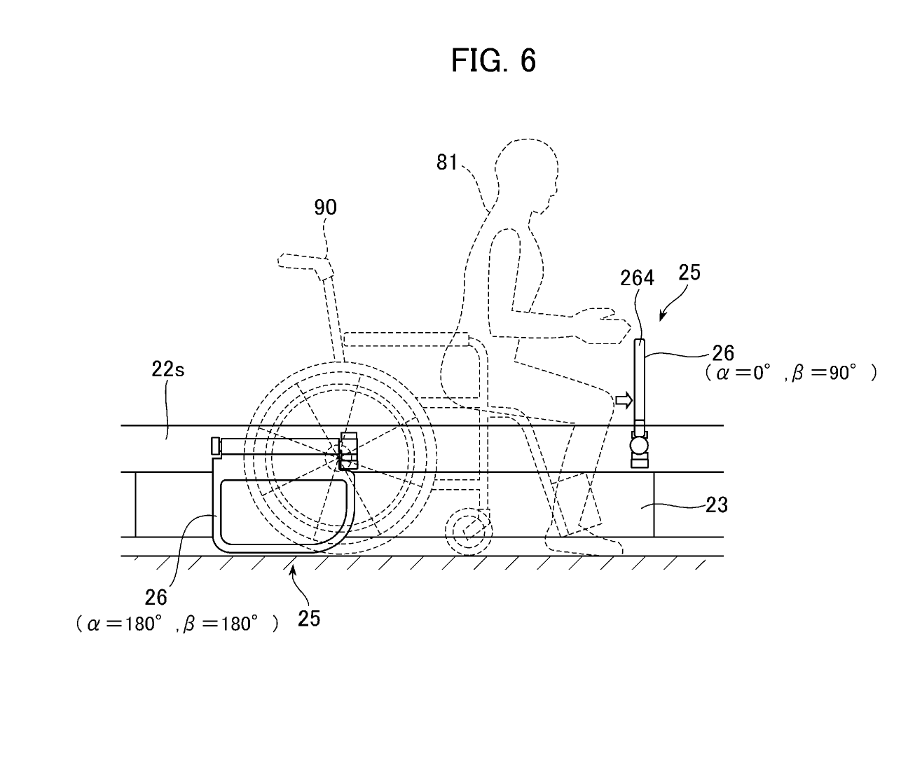

FIG. 6 is a diagram for describing the first function of the pair of door structures 25. For example, as shown in FIG. 6, the table 20 is placed in the lowest position, the door section 26 of one of the right and left door structures 25 is taken to be a horizontal axis rotating angle α=0° and a vertical axis rotating angle β=90° or 135°, and the door section 26 of the other thereof is taken to be a horizontal axis rotating angle α=180° and a vertical axis rotating angle β=180°. That is, the door section 26 of one thereof is locked under the state of being opened to 90° or 135° in the horizontal direction from the home position, whereas the door section 26 of the other thereof is opened at 180° in the horizontal direction from the home position and lowered down by horizontal axis rotation to be locked. In this state, the subject 81 getting on the wheelchair 90 is moved in such a manner that both knees thereof approach the plate-like member 261 or window member 267 of the door section 26 of one thereof. Then, the subject 81 is allowed to take hold of the upper end, i.e., the third columnar member 264 of the door section 26 of one thereof as a grip while abutting both knees against the plate-like member 261 or the window member 267, and then stand up. Thus, the subject 81 is able to greatly reduce a force for supporting the knees during the action of stretching the knees and stably stand up with less ability.

In an embodiment, the height of the knees of the subject 81 standing on the floor from the floor surface is considered to be approximately from about 300 mm to about 400 mm. In embodiments, in order to securely support both knees when the subject 81 stands up from the wheelchair, the door structure 25 is configured in such a manner that when the placement surface of the table 20 is at the lowest position and the first tubular member 271 is at the first horizontal axis rotating position (α=0°), the plate-like member 261 or the window member 267 is positioned in the range in which the height from the floor is 300 mm or more and 500 mm or less.

Taking into consideration easy of use for the subject 81, ease of handling for the operator, space saving and the like, the horizontal width of the door section 26 is 300 mm or more and 500 mm or less.

Second Function: Function of Supporting Getting On/Off the Table

The pair of door structures 25 has as a second function, the function of supporting the operation of the subject 81 getting on and off the table 20.

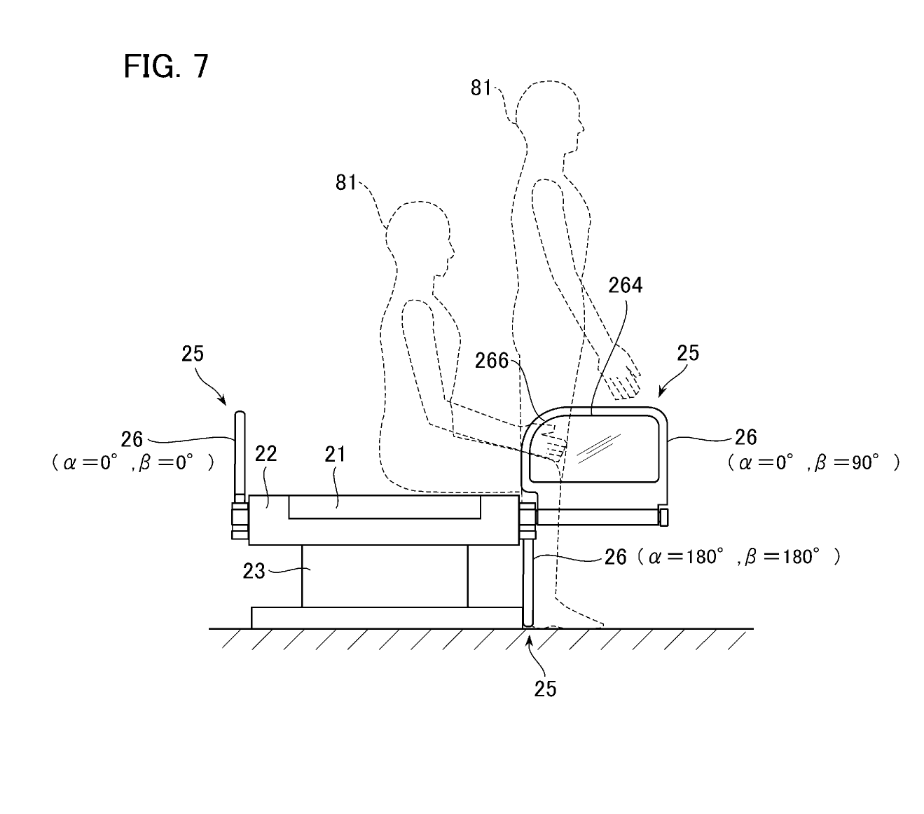

FIG. 7 is a diagram for describing the second function of the pair of door structures 25. For example, as shown in FIG. 7, the table 20 is placed in the lowest position, the door section 26 of one of the right and left door structures 25 is taken to be a horizontal axis rotating angle α=0° and a vertical axis rotating angle β=90° or 135°, and the door section 26 of the other thereof is taken to be a horizontal axis rotating angle α=180° and a vertical axis rotating angle β=180°. That is, the door section 26 of one thereof is locked under the state of being opened to 90° or 135° in the horizontal direction from the home position, whereas the door section 26 of the other thereof is opened at 180° in the horizontal direction form the home position and lowered down to be locked. In this state, the subject 81 getting on the wheelchair or the subject 81 low in athletic ability is allowed to take hold of the third columnar member 264 corresponding to the upper end of the door section 26 of one thereof or the second curved member 266 corresponding to the upper corner part as a grip. Then, the subject 81 is allowed to sit down on the placement surface of the cradle 21. Thereafter, the subject 81 is allowed to place both feet on the cradle 21 so as to stride over the door section 26 of the other thereof. Thus, the subject 81 is able to get on the table 20 without taking an uncomfortable posture. The subject 81 is moved in a procedure opposite to that when the subject 81 gets on even when the subject 81 gets off the table 20, thereby making possible to get off the table 20 without taking the uncomfortable posture.

In an embodiment, the height from the floor surface, of the grip easy to grasp when the subject 81 gets on and off the table 20 is considered to be approximately 500 mm to 800 mm or so. In embodiments, in order to allow the subject 81 to grasp the upper end of the door section 26 as the grip, the door structure 25 is configured in such a manner that when the table 20 is at the lowest position and the first tubular member 271 is at the first horizontal axis rotating position (α=0°), the third columnar member 264 is positioned in the range of the height from the floor surface, which is 500 mm or more and 800 mm or less. The thickness of the grip easy to grasp by the subject 81 is considered to be approximately from about 30 mm to about 50 mm in diameter. In embodiments, in order to allow the subject 81 to easily grasp the upper end of the door section 26 as the grip, the door structure 25 is configured in such a manner that the thickness of the third columnar member 264 becomes 30 mm or more and 50 mm or less in diameter.

In an embodiment, as described above, the pair of door structures 25 is provided in such a manner as to be bilaterally symmetric with each other in the center in the z-axis direction of the side face 22s of the cradle support body 22. Therefore, even when the direction in which the head of the subject 81 is oriented on the table 20 is made opposite, the first and second functions can cope with it by simply reversing the state of the right and left door structures 25.

Third Function: Function of Supporting Getting-Up on the Table

The pair of door structures 25 has as a third function the function of supporting the action of the subject 81 getting up on the table 20.

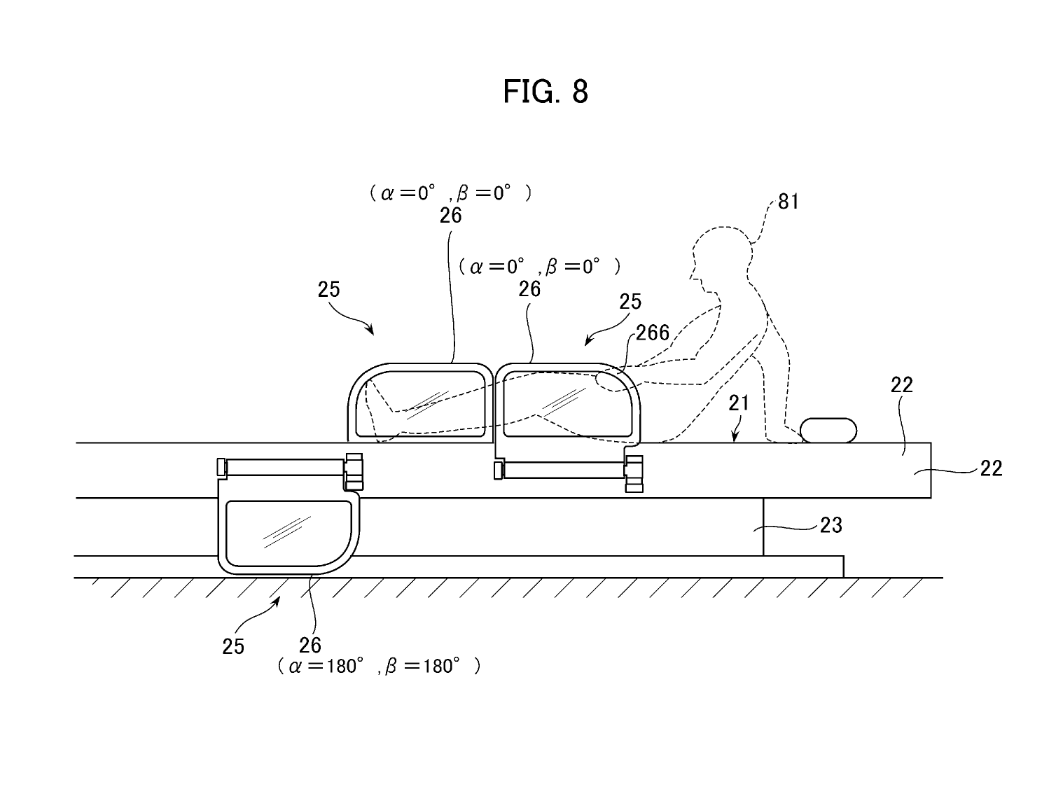

FIG. 8 is a diagram for describing the third function of the pair of door structures 25. As shown in FIG. 8, for example, when the subject 81 is in a state of being placed on the cradle 21 of the table 20, the door section 26 on the side close to at least the head of the subject 81, of the right and left door structures 25 is set to the home position and then locked. Since the door section 26 should be in a state of being opened in the horizontal direction from the home position if it is the right after the subject 81 has gotten on the table 20, the door section 26 may be returned to the original. When the subject 81 assumes a lying-down posture (laid posture) from the posture in which the subject 81 sits on the placement surface of the cradle 21 with the buttocks being placed on the placement surface, and reversely when the subject 81 stands up from the lying-down posture and assumes the posture in which the subject 81 sits on the placement surface of the cradle 21 with the buttocks being placed on the placement surface, the subject 81 is allowed to grasp the second curved member 266 of the door section 26 on the side close to the head as a grip. Thus, the subject 81 is able to disperse the load applied to the upper body thereof and get up relatively easily.

In an embodiment, the shape of the grip easy to grasp when the subject 81 gets up on the table 20 is considered to be a curved shape of which the radius of curvature is approximately from about 150 mm to about 300 mm. Thus, in order to allow the subject 81 to easily grasp the upper corner part of the door section 26 as a grip when the subject 81 gets up on the table 20, in an embodiment of the present invention, the door structure 25 is configured such that the second curved member 266 is curved with a radius of curvature that is 150 mm or more and 300 mm or less, for example. Since the thickness of the grip easy for the subject 81 to grasp is considered to be approximately from about 30 mm to about 50 mm in diameter, in embodiments, the second curved member 266 is 30 mm or more and 50 mm or less in diameter.

Fourth Function: Function of Preventing Falling Form the Table

The pair of door structures 25 has as a fourth function, the function of preventing the subject 81 from falling from the table 20.

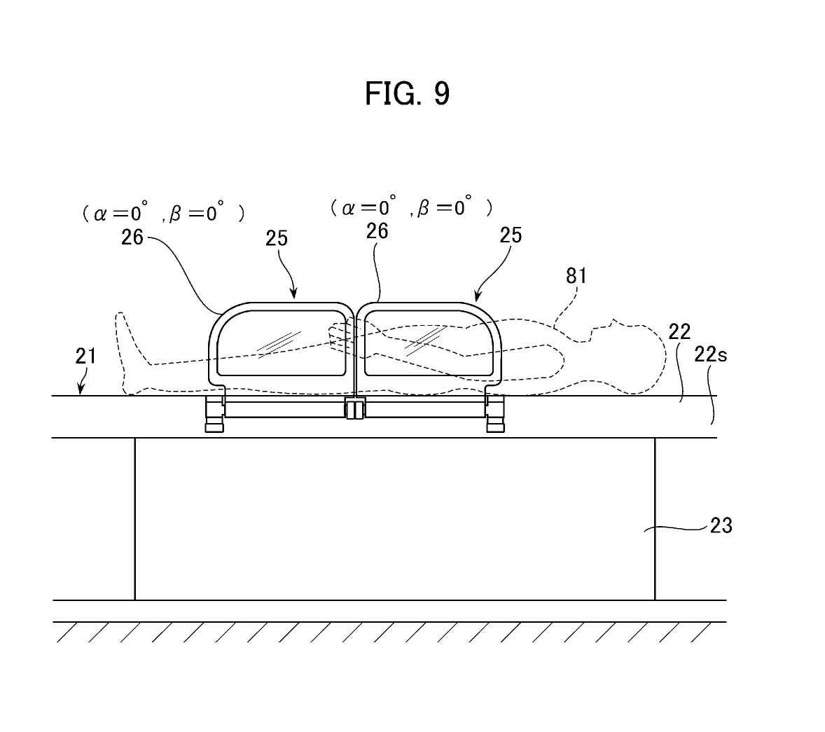

FIG. 9 is a diagram for describing the fourth function of the pair of door structures 25. As shown in FIG. 9, for example, when the subject 81 is in a state of being placed on the cradle 21 of the table 20 or in a sleeping state, the door sections 26 of both of the right and left door structures 25 are placed in the home position and locked. Thus, since the subject 81 is held not to protrude outside from the cradle 21 even when the subject 81 is laterally moved, it is possible to prevent the subject 81 from falling. Taking the fall-prevention measures by the door structures 25 enables giving of a sense of security to the subject 81.

In an embodiment, when a subject 81 such as a subject 81 high in athletic ability and capable of walking is transferred between a stretcher and a table, there is a possibility that the door structures 25 will result in an obstacle to getting on and off the table 20 after all. In such a case, the pair of door structures 25 lying on the getting on/off side may be placed in a state of the horizontal axis rotating angle α=180° and the vertical axis rotating angle β=180° respectively to prevent the pair of door structures 25 from becoming obstacles to the getting on and off of the subject 81 on the table 20. That is, the right and left door sections 26 may respectively be opened in the horizontal direction from their home positions and moved down to be brought to a locked state.

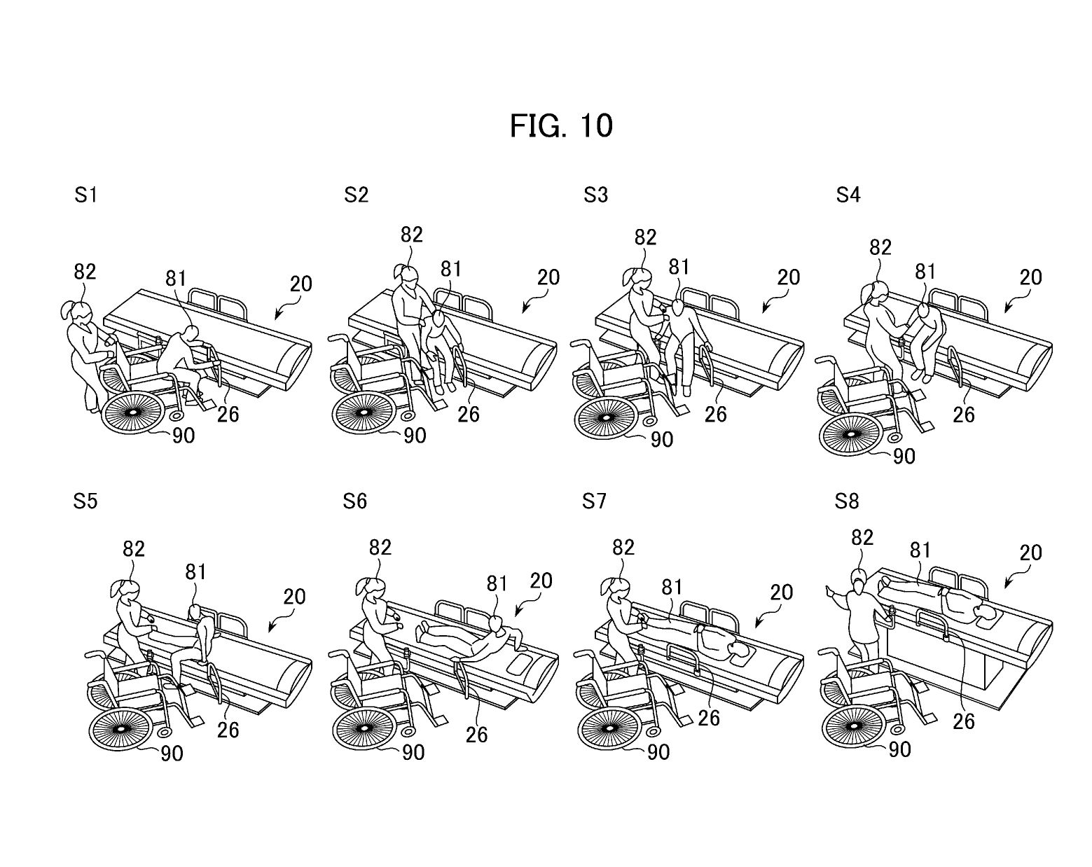

FIG. 10 is a diagram showing the flow of movements of the subject 81 getting on the wheelchair 90 and the operator when the subject 81 is placed on the table 20.

At step S1, the operator 82 opens in advance the door section 26 of one of the right and left door structures 25 of the table 20, which is on the side in the direction in which the head of the subject 81 is placed, by about 90° to 135° from the home position by vertical axis rotation, thereby locking it. The door section 26 of the other thereof is opened at 180° in the horizontal direction from the home position by vertical axis rotation and further opened at 180° by horizontal axis rotation and lowered down to be locked. The operator 82 guides the subject 81 placed on the wheelchair 90 to the front of the door section 26 of above one. Then, the subject 81 is allowed to apply both knees onto the door surface of the door section 26 of above one as needed. The subject 81 is allowed to grasp the upper end of the door section 26 and then stand up.

At step S2, the subject 81 is allowed to sit down on the table 20 while grasping the upper end and upper corner part of the door section 26 of above one. The operator 82 assists its motion.

At step S3, the subject 81 is allowed to adjust the direction of his/her body and then sit down on the table 20 in a stable posture. The operator 82 assists the motion of the subject 81.

At step S4, the subject 81 is allowed to move his/her hip part to the center of the table 20.

At step S5, the subject 81 is allowed to put both feet on the table 20 over the door section 26 of the other of the door structures 25.

At step S6, the operator 82 closes the door section 26 of above one to bring it back to the home position. Then, the subject 81 is allowed to bend the upper body back while grasping the upper corner part of the door section 26 of above one.

At step S7, the subject 81 is allowed to completely lie down on the table 20. The operator 82 confirms the position of the subject 81 and finely adjusts the position as needed.

At step S8, the operator 82 operates the table 20 to raise it to a suitable height.

According to the present embodiment as described above, the door structures 25 each having the door section 26, and the horizontal axis rotation lock mechanism 27 and the vertical axis rotation lock mechanism 28 are provided at each side face 22s of the table 20. Therefore, the door section 26 is opened horizontally by the vertical axis rotation and locked with respect to the subject 81 getting on the wheelchair 90 or the subject 81 low in athletic ability due to the old age or disorders to thereby enable the subject 81 to get on and off the table 20 while grasping the door section 26. On the other hand, for the subject 81 high in athletic ability, the door section 26 is opened horizontally by the vertical axis rotation and lowered down by the horizontal axis rotation, thereby making it possible to get the subject 81 on and off the table 20 without being disturbed by the door section 26. Thus, the table 20 can be deformed according to the athletic ability of the subject 81 to make the subject 81 easy to get on and off the table 20. As a result, the subject 81 becomes easy to get on and off the table 20 regardless of being high or low in athletic ability.

In the present embodiment as well, even when the subject 81 is transferred between the stretcher and the table, the door section 26 is opened horizontally by the vertical axis rotation and lowered down by the horizontal axis rotation, thereby making it possible to transfer the subject 81 therebetween without being disturbed by the door section 26.

Further, in the present embodiment, the operator 82 is also able to reduce the amount of support of the motion of the subject 81. The operator 82 and the subject 81 are both able to reduce a physical burden.

Furthermore, in the present embodiment, since the efficiency of the operation of allowing the subject 81 to get on and off the table 20 is improved, it is possible to shorten an inspection time and improve test productivity.

Still further, in the present embodiment, the plate-like member 261 or window member 267 of each of the door sections 26 is configured to be placed at the height of the knees of the subject 81 standing on the floor. Thus, in particular, a subject 81 on a wheelchair is able to stand up while his/her knees are being applied onto the plate-like member 261 or the window member 267 when the subject 81 stands up from the wheelchair. Therefore, the subject 81 on the wheelchair is able to greatly reduce a force for supporting the knees during the action of stretching the knees at his/her stand-up and stably stand up with less ability.

Still further, in the present embodiment, when the subject high in athletic ability gets on and off, the door section 26 can be opened horizontally (laterally) and lowered. Therefore, the door section 26 does not get in the way even though being at the feet of the subject. There is no need to detach the door structures 25 as in the related art.

Still further, in the present embodiment, since the door structures 25 are provided at both side faces 22s of the cradle support body 22, the door structures 25 can be adapted even if the side of the subject 81 getting on and off the table 20 is brought to either of both side faces 22 according to the conditions of a facility.

Still further, in the present embodiment, since the horizontal axis rotation lock mechanism 27 is capable of locking each door section 26 at the third horizontal axis rotating position where the door section 26 becomes horizontal, the door section 26 can be utilized as a location to place equipment or the like when it is desired to place the equipment or the like beside the subject 81.

Still further, in the present embodiment, since the window member 267 mentioned above is provided and no opening is formed in the door section 26, it is possible to prevent an accident such that the arm or the like of the subject 81 accidentally enters the opening and is caught therein when the cradle 21 is linearly moved horizontally.

Still further, in the present embodiment, since the window member 267 is comprised of a member having light transmission properties, the subject 81 is able to look outside through the window member 267 even when the subject 81 is placed on the table 20 and the door section 26 is in home position, thus making it possible to prevent a feeling of pressure to the subject 81.

Still further, in the present embodiment, since each of the door structures 25 serves not only as a handrail to grasp upon getting on/off but also as a role for falling prevention, and always supports the subject 81, the subject 81 is able to relax tension and a feeling of fear and reduce a mental burden.

In the present embodiment, although the pairs of door structures 25 are provided on both side faces 22s of the cradle support body 22, other types of door structures may be provided on either one of the side faces 22s. Further, the door structures 25 are not necessarily required to be provided in a left and right pair and may singly be provided.

Since the optimum sizes of the respective parts constituting the door section 26 differ depending on countries and human races, they may be adjusted according to these. The size employed in the present embodiment is intended for use in Asian countries. Thus, for example, the size may be adjusted to 1.2 to 1.5 times the size in the present embodiment in the U.S. and European countries.

Although the present embodiment shows the example in which the invention has been applied to the table for the magnetic resonance imaging system, the invention can be applied even to tables for other medical equipment such as a radiation tomographic imaging apparatus, a radiation therapy apparatus, etc.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A medical equipment table having a placement surface on which a subject is placed, comprising:
door structures provided at side faces extending along a longitudinal direction of the medical equipment table,
wherein each of the door structures includes:
a door section;
a horizontal axis rotation lock mechanism enabling horizontal axis rotation with one end in a vertical direction of the door section being taken as a support axis, and locks at a plurality of horizontal axis rotating positions; and
a vertical axis rotation lock mechanism enabling vertical axis rotation with one end in a horizontal direction of the door section being taken as a support axis, and locks at a plurality of vertical axis rotating positions.

2. The medical equipment table according to claim 1,
wherein the horizontal axis rotating positions include a horizontal axis rotating position where the door section is directed upward with respect to the support axis for the horizontal axis rotation and a horizontal axis rotating position where the door section is directed downward with respect to the support axis for the horizontal axis rotation, and
wherein the vertical axis rotating positions include a first vertical axis rotating position where an opening angle of the door section to the side face becomes substantially 0°, and a second vertical axis rotating position where the opening angle becomes substantially larger than 0° and equal to or smaller than 180°.

3. The medical equipment table according to claim 1, wherein the door structures are provided in a pair in the center in the longitudinal direction of the table so as to be left and right symmetrical.

4. The medical equipment table according to claim 1, wherein the door structures are respectively provided at both side faces extending along the longitudinal direction of the table.

5. The medical equipment table according to claim 2, including an elevation section which elevates the placement surface,
wherein the door section has a plate-like member placed in a position where a height from a floor becomes 300 mm or more and 400 mm or less when the placement surface is at a lowermost position and the door section is at an upward horizontal axis rotating position.

6. The medical equipment table according to claim 2, wherein the horizontal axis rotating positions include a horizontal axis rotating position where the door section becomes horizontal with respect to the support axis for the horizontal axis rotation.

7. The medical equipment table according to claim 5, wherein each of the door structures is configured in such a manner that when the placement surface is at the lowermost position and the door section is at the upward horizontal axis rotating position, a height from the floor to an upper end of the door section becomes 500 mm or more and 800 mm or less.

8. The medical equipment table according to claim 5, wherein the door section is formed in such a manner that one of upper corner parts section at the upward horizontal axis rotating position becomes a curved shape which is or more and 300 mm or less in the radius of curvature.

9. The medical equipment table according to claim 1, wherein the door section is formed in such a manner that a horizontal width thereof becomes 300 mm or more and 500 mm or less.

10. The medical equipment table according to claim 1,
wherein the horizontal axis rotation lock mechanism has a first tubular member connected to one end in the vertical direction of the door section and extending in the horizontal direction, a first fitting member inserted from an opening at one end of the first tubular member and fitted into the first tubular member, and a first engagement member inserted from an opening at the other end of the first tubular member and engaged with the first tubular member and the first fitting member within the cylinder of the first tubular member, and
wherein the vertical axis rotation lock mechanism has a second tubular member connected to one end in the horizontal direction of the first fitting member and extending in the vertical direction, a second fitting member inserted from an opening at one end of the second tubular member and fitted into the second tubular member, a second engagement member inserted from an opening at the other end of the second tubular member and engaged with the second tubular member and the second fitting member within the cylinder of the second tubular member, and a support portion which is fixed to the side face and supports the second fitting member.

11. The medical equipment table according to claim 10, wherein horizontal axis rotation lock mechanism and the vertical axis rotation lock mechanism respectively have a plurality of convex portions formed in a circumferential direction and plurality of concave portions formed in the circumferential direction and fitted onto the portions.

12. Medical equipment having a medical equipment table according to claim 1.

13. The medical equipment according to claim 12, which is configured to perform magnetic resonance imaging of the subject.

14. The medical equipment according to claim 13, wherein the door structure is comprised of a nonmagnetic body.

15. The medical equipment according to claim 12, which is configured to perform radiation tomography of the subject.

16. The medical equipment according to claim 12, which is configured to perform radiation therapy of the subject.

17. The medical equipment table according to claim 2, wherein the door structures are provided in a pair in the center in the longitudinal direction of the table so as to be left and right symmetrical.

18. The medical equipment table according to claim 2, wherein the door structures are respectively provided at both side faces extending along the longitudinal direction of the table.

19. The medical equipment table according to claim 3, wherein the structures are respectively provided at both side faces extending along the longitudinal of the table.

20. The medical equipment table according to claim 5, wherein the horizontal axis rotating positions include a horizontal axis rotating position where the door section becomes horizontal with respect to the support axis for the horizontal axis rotation.
